(19) United States
(12) Patent Application Publication
(10) Pub. No.: US 2007/0265329 A1
(43) Pub. Date: Nov. 15, 2007

(54) Methods for the prevention of acute and delayed chemotherapy-induced nausea and vomiting (CINV)

(76) Inventors: Shah T. Devang, Redwood City, CA (US); John Barr, Redwood City, CA (US); Brian Baxter, Redwood City, CA (US); Jorge Heller, Ashland, OR (US)

Correspondence Address: HELLER EHRMAN LLP, 275 MIDDLEFIELD ROAD, MENLO PARK, CA, 94025-3506, US

(21) Appl. No.: 11/433,917

(22) Filed: May 12, 2006

(51) Int. Cl. A61K 31/405

(52) U.S. Cl. 514/419

(57) ABSTRACT

A pharmaceutical composition for the sustained release of an effective amount of a selective 5-hydroxytryptamine 3 (5-HT₃) receptor antagonist for the prevention, reduction or alleviation of acute and delayed chemotherapy-induced nausea and vomiting (CINV) following a course of emetogenic chemotherapy, wherein the composition is administered by subcutaneous injection, the composition comprising a 5-HT₃ receptor antagonist, a semi-solid delivery vehicle and a pharmaceutically acceptable liquid excipient; wherein the composition, when administered in a single dosage, provides a controlled release of the 5-HT₃ receptor antagonist and prolonging the release of the 5-HT₃ receptor antagonist that tracks the profile of the incidence of vomiting.

### BACKGROUND OF THE INVENTION

1. Field of the Invention

This invention relates to novel methods and protocols of administration of semi-solid delivery vehicles comprising a polyorthoester and an excipient, and to controlled release pharmaceutical compositions comprising the delivery vehicle and a 5-HT₃ antagonist such as granisetron. The pharmaceutical compositions may be in the form of a syringable or injectable formulation for local controlled delivery of the active agent.

2. Description of the Art

One of the side-effects most feared by patients undergoing chemotherapy treatment is nausea and vomiting (CINV). In recent years supportive care products to treat the side-effects of chemotherapy, such as CINV, have emerged to improve patient comfort and compliance with treatment regiments. The advent of selective 5-hydroxytryptamine 3 (5-HT₃) antagonists has revolutionized the management of nausea and vomiting experience by cancer patients undergoing chemotherapy. Nausea and vomiting patterns are classified as acute or delayed. Approximately 95% of patients receiving chemotherapy will experience some degree of emesis if not prevented with an antiemetic. If untreated, CINV is estimated to affect 85% of cancer patients undergoing chemotherapy and can result in delay or even discontinuation of treatment. And if emesis is not properly managed, it can cause dehydration and poor quality of life, and may eventually lead to interruption or discontinuation of chemotherapy.

CINV patterns are classified as either acute or delayed. Acute onset CINV usually occurs within minutes or a few hours of receiving chemotherapy. The symptoms peak after about 6 hours and can last for approximately 24 hours. Delayed onset CINV is an episode of nausea and vomiting that occurs after 24 hours of administration of chemotherapy and can last for several days. Delayed onset CINV can significantly and adversely affect a patient's ability to provide self care once the patient has been discharged from the hospital.

Clinicians routinely include antiemetic agents as part of chemotherapy regimes; but according to recent surveys it has been demonstrated that healthcare providers underestimate the frequency and severity of delayed nausea and vomiting after chemotherapy. There is a significant need for a long-acting anti-emetic agent that could be conveniently delivered prior to the administration of chemotherapy and that could prevent both acute and delayed emesis with a single dose, and also providing optimal therapeutic effects while minimizing any undesired side effects resulting from the treatment.

There are four 5-HT₃ antagonists currently on the U.S. market: Kytril® (granisetron), Zofran® (ondansetron), Anzemet® (dolasetron) and Aloxi® (palonosetron, MGI Pharma). All 5-HT₃ antagonists are equally effective in the prevention of chemotherapy-induced nausea and vomiting in the acute phase (0-24 hours). Only Aloxi®, a 5-HT₃ antagonist with a longer half-life, is currently approved for the prevention of delayed nausea and vomiting (24-120 hours) with initial and repeat courses of moderately emetogenic cancer therapy. In clinical studies, the majority of patients taking Aloxi® did not vomit or need additional medication for nausea on the first day (72%) and on days 2-5 (64%) following moderately emetogenic chemotherapy. However, it is well established that the most common side effects associated with patients taking Aloxi® are headache and constipation.

The above 5-HT₃ antagonists that are used commercially have been shown to reduce the incidence and severity of acute nausea and vomiting, but the short half-lives of these initial products, or the undesired side effects limited their effectiveness against delayed onset CINV. Therefore, there is an urgent need for the development of a long-acting anti-emetic agent that would provide relief over three to five days without the undesired side effects.

A large class of active agents such as antibiotics, antiseptics, corticosteroids, anti-neoplastics, and local anesthetics may be administered to the skin or mucous membrane by topical application, or by injection. Topical delivery may be accomplished through the use of compositions such as ointments, creams, emulsions, solutions, suspensions and the like. Injections for delivery of the active agents include solutions, suspensions and emulsions. All of these preparations have been extensively used for delivery of active agents for years. However, these preparations suffer the disadvantage that they are short-acting and therefore they often have to be administered several times in a day to maintain a therapeutically effective dose level in the blood stream at the sites where the activity/treatment is required.

In recent years, a great deal of progress has been made to develop dosage forms which, after their administration, provide a long-term therapeutic response. These products may be achieved by microencapsulation, such as liposomes, microcapsules, microspheres, microparticles and the like. For this type of dosage forms, the active agents are typically entrapped or encapsulated in microcapsules, liposomes or microparticles which are then introduced into the body via injection or in the form of an implant. The release rate of the active agent from this type of dosage forms is controlled which eliminates the need for frequent dosing.

### SUMMARY OF THE INVENTION

In one aspect of the invention, there is provided a pharmaceutical composition for the sustained and controlled release of an effective amount of a selective 5-hydroxytryptamine 3 (5-HT₃) receptor antagonist for the prevention, reduction or alleviation of acute and delayed chemotherapy-induced nausea and vomiting (CINV) following a course of emetogenic chemotherapy, wherein the composition is administered by subcutaneous injection, the composition comprising a 5-HT₃ receptor antagonist, a semi-solid delivery vehicle and a pharmaceutically acceptable liquid excipient; wherein the composition, when administered in a single dosage, provides the release profile of the 5-HT₃ receptor antagonist that tracks the profile of the incidence of vomiting.

In another aspect, there is provided methods of using the above pharmaceutical compositions for the prevention, reduction or alleviation of acute and delayed chemotherapy-induced nausea and vomiting (CINV).

### ASPECTS OF THE INVENTION

In one aspect, there is provided a pharmaceutical composition for the sustained and controlled release of an effective amount of a selective 5-hydroxytryptamine 3 (5-HT₃) receptor antagonist for the prevention, reduction or alleviation of acute and delayed chemotherapy-induced nausea and vomiting (CINV) following a course of emetogenic chemotherapy, wherein: the composition is administered by subcutaneous injection, the composition comprising a 5-HT₃ receptor antagonist, a semi-solid delivery vehicle and a pharmaceutically acceptable liquid excipient; wherein the composition, when administered in a single dosage, provides a release profile of the 5-HT₃ receptor antagonist that tracks the profile of an incidence of vomiting; and wherein the composition provides a level of the 5-HT₃ receptor antagonist over 24 hours to provide a % Cmax profile that is within 20% of the profile in the incidence of vomiting, provides a sustained levels over 96 hours to provide a % Cmax profile that is within 10% of the profile in the incidence of vomiting, and provides essentially no 5-HT₃ receptor antagonist concentration in plasma at about 144 hours. In a variation of the above composition, the level of the 5-HT₃ receptor antagonist over 24 hours, is substantial and may be measured experimentally as provided herein, to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no detectable 5-HT₃ receptor antagonist concentration in plasma at about 144 hours.

In one variation of the above pharmaceutical composition, the 5-HT₃ receptor antagonist is granisetron. In another variation, the effective amount of the 5-HT₃ receptor antagonist is a single dose of about 5 mg to about 10 mg. In another variation, the administration by subcutaneous injection is performed at about three hours, two hours, one hour, or 30 minutes before chemotherapy. In a particular variation of the above, the administration by subcutaneous injection is performed at about 30 minutes before chemotherapy. In another variation, the subcutaneous injection is performed over about 30 seconds. In yet another variation, the 5-HT₃ receptor antagonist is granisetron and the effective amount of granisetron is about 5 mg.

In another aspect of the above, the composition provides a substantial level of the 5-HT₃ receptor antagonist over 24 hours to provide a % Cₘₐₓ profile that is within 10% of the profile in the incidence of vomiting. In another variation, the composition provides sustained levels over 96 hours to provide a % Cₘₐₓ profile that is within 5% of the profile in the incidence of vomiting. In another particular variation, the composition a substantial level of the 5-HT₃ receptor antagonist over 24 hours to provide a % Cₘₐₓ profile that is within 10% of the profile in the incidence of vomiting, provides a sustained levels over 96 hours to provide a % Cₘₐₓ profile that is within 5% of the profile in the incidence of vomiting, and provides essentially no 5-HT₃ receptor antagonist concentration in plasma at about 144 hours. In another variation, the administration of an effective amount of the 5-HT₃ receptor antagonist to a patient result in further reducing the incidence of reported headaches to less than about 40%, 30%, 20% or about 10% in patients receiving chemotherapy. In another variation of the above, the incidence of reported headaches is less than about 20% in patients receiving chemotherapy.

In another aspect, there is provided a pharmaceutical composition for the sustained and controlled release of an effective amount of a selective 5-hydroxytryptamine 3 (5-HT₃) receptor antagonist for the prevention, reduction or alleviation of acute and delayed chemotherapy-induced nausea and vomiting (CINV) following a course of emetogenic chemotherapy, wherein: the composition is administered by subcutaneous injection, the composition comprising a 5-HT₃ receptor antagonist, a semi-solid delivery vehicle and a pharmaceutically acceptable liquid excipient; wherein:

(A) the semi-solid delivery vehicle, comprises:

(i) a polyorthoester of formula I, formula II, formula III or formula IV

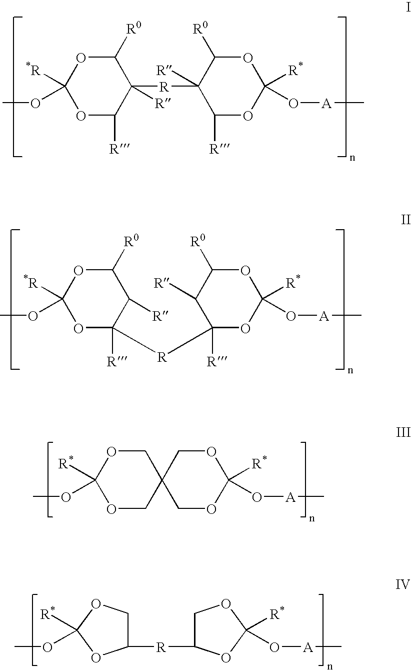

where:

R is a bond, —(CH₂)ₐ—, or —(CH₂)_{b}—O—(CH₂)_{c}—; where a is an integer of 1 to 10, and b and c are independently integers of 1 to 5;

R* is a C₁₋₄ alkyl;

R^{o}, R″ and R′″ are each independently H or C₁₋₄ alkyl;

n is an integer of at least 5; and

A is R¹, R², R³, or R⁴, where

R¹ is: 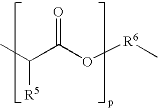
where:

p is an integer of 1 to 20;

R⁵ is hydrogen or C₁₋₄ alkyl; and

R⁶ is: 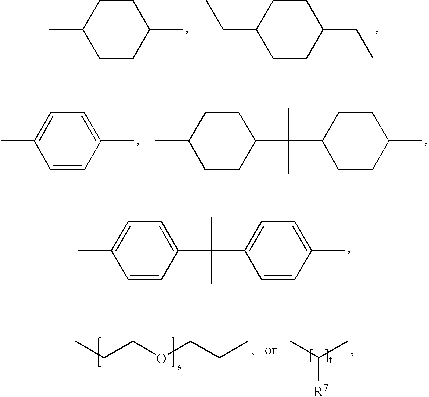
where:

s is an integer of 0 to 30;

t is an integer of 2 to 200; and

R⁷ is hydrogen or C₁₋₄ alkyl,

R² is: 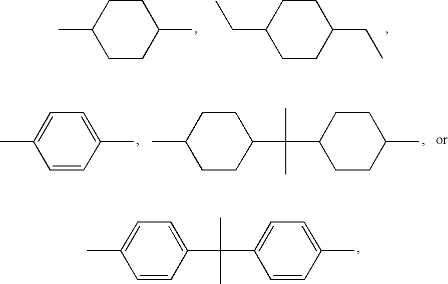
R³ is: 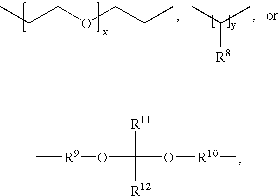
where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R¹¹ and R¹² together are C₃₋₁₀ alkylene; and

R⁴ is the residue of a diol containing at least one functional group independently selected from amide, imide, urea, and urethane groups;

in which at least 0.01 mol percent of the A units are of the formula R¹; and

(ii) a pharmaceutically acceptable, polyorthoester-compatible liquid excipient selected from polyethylene glycol ether derivatives having a molecular weight between 200 and 4000, polyethylene glycol copolymers having a molecular weight between 400 and 4000, mono-, di-, or tri-glycerides of a C₂₋₁₉ aliphatic carboxylic acid or a mixture of such acids, alkoxylated tetrahydrofurfuryl alcohols and their C₁₋₄ alkyl ethers and C₂₋₁₉ aliphatic carboxylic acid esters, and biocompatible oils; and

(B) wherein the 5-HT₃ receptor antagonists is granisetron; wherein the composition, when administered in a single dosage, provides the release profile of the 5-HT₃ receptor antagonist that tracks the profile of the incidence of vomiting, and wherein the composition provides a substantial level of the 5-HT₃ receptor antagonist over 24 hours to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no 5-HT₃ receptor antagonist concentration in plasma at about 144 hours.

In one variation of the above composition, A is R¹, R³, or R⁴, where

R¹ is:

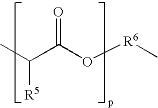

where:

p is an integer of 1 to 20;

R³ and R⁶ are each independently:

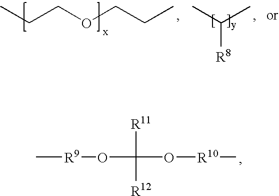

where:

x is an integer of 0 to 30;

y is an integer of 2 to 200;

R⁸ is hydrogen or C₁₋₄ alkyl;

R⁹ and R¹⁰ are independently C₁₋₁₂ alkylene;

R¹¹ is hydrogen or C₁₋₆ alkyl and R¹² is C₁₋₆ alkyl; or R¹¹ and R¹² together are C₃₋₁₀ alkylene;

R⁴ is a diol containing at least one functional group independently selected from amide, imide, urea, and urethane groups; and R⁵ is hydrogen or C₁₋₄ alkyl; and in which at least 0.01 mol percent of the A units are of the formula R¹.

In another variation, the concentration of the polyorthoester ranges from 1% to 99% by weight. In yet another variation, the polyorthoester has a molecular weight between 3,000 and 10,000. In a particular variation, the fraction of the A units that are of the formula R¹ is between 5 and 15 mole percent.

In another variation of the above pharmaceutical composition, the polyorthoester is of formula I, where: none of the units have A equal to R²;

R³ is: 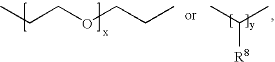
where:

x is an integer of 0 to 10;

y is an integer of 2 to 30; and

R⁶ is: 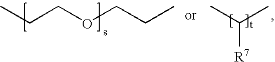
where:

s is an integer of 0 to 10;

t is an integer of 2 to 30; and

R⁵, R⁷, and R⁸ are independently hydrogen or methyl.

In a particular variation of the above cited composition, R³ and R⁶ are both —(CH₂—CH₂—O)₂—(CH₂—CH₂)—; R⁵ is methyl; and p is 1 or 2. In another variation, R³ and R⁶ are both —(CH₂—CH₂—O)₉—(CH₂—CH₂)—; R⁵ is methyl; and p is 1 or 2. In a particular variation of the above, the fraction of granisetron is from 0.1% to 80% by weight of the composition. In yet another variation, the fraction of granisetron is about 1% to 5% by weight of the composition. In another variation, the polyorthoester is of formula I, R is —(CH₂)_{b}—O—(CH₂)_{c}—; where b and c are both 2; R* is a C₂ alkyl; where the excipient is methoxy-polyethylene glycol (Mn 550), and granisetron comprises 2 wt % of the composition.

In another embodiment, there is provided a method for the prevention, reduction or alleviation of acute and delayed chemotherapy-induced nausea and vomiting (CINV) following a course of emetogenic chemotherapy, the method comprising: administering a single dosage of a pharmaceutical composition for the sustained and controlled release of an effective amount of a selective 5-hydroxytryptamine 3 (5-HT₃) receptor antagonist by subcutaneous injection; wherein the composition comprises a 5-HT₃ receptor antagonist, a semi-solid delivery vehicle and a pharmaceutically acceptable liquid excipient, wherein the method provides the release profile of the 5-HT₃ receptor antagonist that tracks the profile of the incidence of vomiting, and wherein the method provides a substantial level of the 5-HT₃ receptor antagonist over 24 hours to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no 5-HT₃ receptor antagonist concentration in plasma at about 144 hours. In one variation, the 5-HT₃ receptor antagonist is granisetron. In another variation, the effective amount of the 5-HT₃ receptor antagonist is a single dose of about 5 mg. In another variation, the administration by subcutaneous injection is performed at about three hours, two hours, one hour, or 30 minutes before chemotherapy. In yet another variation, the administration by subcutaneous injection is performed at about 30 minutes before chemotherapy. In yet another variation, the subcutaneous injection is performed over about 30 seconds.

In a particular variation of the above, the 5-HT₃ receptor antagonist is granisetron and the effective amount of granisetron is about 5 mg. In another variation of the above method, the composition provides a substantial level of the 5-HT₃ receptor antagonist over 24 hours to provide a % Cₘₐₓ profile that is within 10% of the profile in the incidence of vomiting. In yet another variation, the composition provides sustained levels over 96 hours to provide a % Cₘₐₓ profile that is within 5% of the profile in the incidence of vomiting.

In another aspect, there is provided the above noted pharmaceutical composition, wherein the composition a substantial level of the 5-HT₃ receptor antagonist over 24 hours to provide a % Cₘₐₓ profile that is within 10% of the profile in the incidence of vomiting, provides a sustained levels over 96 hours to provide a % Cₘₐₓ profile that is within 5% of the profile in the incidence of vomiting, and provides essentially no 5-HT₃ receptor antagonist concentration in plasma at about 144 hours. In one variation, the administration of an effective amount of the 5-HT₃ receptor antagonist to a patient result in further reducing the incidence of reported headaches to less than about 40%, 30%, 20% or about 10% in patients receiving chemotherapy. In another variation, the incidence of reported headaches is less than about 20% in patients receiving chemotherapy. In yet another variation, the administration of an effective amount of the 5-HT₃ receptor antagonist to a patient result in a statistically significant reduction in the incidence of vomiting following emetogenic chemotherapy than that of the administration of palonosetron by iv infusion.

### DETAILED DESCRIPTION OF THE INVENTION

Definitions

Unless defined otherwise in this specification, all technical and scientific terms are used herein according to their conventional definitions as they are commonly used and understood by those of ordinary skill in the art of synthetic chemistry and pharmacology.

“Bioerodible” and “bioerodibility” refer to the degradation, disassembly or digestion of the polyorthoester by action of a biological environment, including the action of living organisms and most notably at physiological pH and temperature. A principal mechanism for bioerosion of the polyorthoesters of the present invention is hydrolysis of linkages between and within the units of the polyorthoester.

“Comprising” is an inclusive term interpreted to mean containing, embracing, covering or including the elements listed following the term, but not excluding other unrecited elements.

“Sustained release”, “extended release” and similar terms are used to denote a mode of active agent delivery that occurs when the active agent is released from the delivery vehicle at an ascertainable and controllable rate over a period of time, rather than dispersed immediately upon application or injection. Extended or sustained release, which may also be controlled using the methods taught in the present invention, may extend for hours, days or months, and may vary as a function of numerous factors. For the pharmaceutical composition of the present invention, the rate of release will depend on the type of the excipient selected and the concentration of the excipient in the composition. Another determinant of the rate of release is the rate of hydrolysis of the linkages between and within the units of the polyorthoester. The rate of hydrolysis in turn may be controlled by the composition of the polyorthoester and the number of hydrolyzable bonds in the polyorthoester. Other factors determining the rate of release of granisetron from the present pharmaceutical composition include particle size, solubility of the active agent, acidity of the medium (either internal or external to the matrix) and physical and chemical properties of the active agent in the matrix. As used herein, “controlled release” used in combination with “sustained release” of the pharmaceutical composition also means that the specific profile of the release of the active agent, such as granisetron, in addition to the sustained or extended release period, may be controlled to provide optimum efficacy with the desired therapeutic effects.

“Delivery vehicle” denotes a composition which has the functions including transporting an active agent to a site of interest, controlling the rate of access to, or release of, the active agent by sequestration or other means, and facilitating the application of the agent to the region where its activity is needed.

“Polyorthoester-compatible” refers to the properties of an excipient which, when mixed with the polyorthoester, forms a single phase and does not cause any physical or chemical changes to the polyorthoester.

“Semi-solid” denotes the mechano-physical state of a material that is flowable under moderate stress. More specifically, the semi-solid material should have a viscosity between about 10,000 and 3,000,000 cps, especially between about 30,000 and 500,000 cps. Preferably the formulation is easily syringable or injectable, meaning that it can readily be dispensed from a conventional tube of the kind well known for topical or ophthalmic formulations, from a needleless syringe, or from a syringe with a 16 gauge or smaller needle, such as 16-25 gauge.

“Sequestration” is the confinement or retention of an active agent within the internal spaces of a polyorthoester matrix. Sequestration of an active agent within the matrix may limit the toxic effect of the agent, prolong the time of action of the agent in a controlled manner, permit the release of the agent in a precisely defined location in an organism, or protect unstable agents against the action of the environment.

A “therapeutically effective amount” means the amount that, when administered to an animal for treating a disease, is sufficient to effect treatment for that disease.

“Treating” or “treatment” of a disease includes preventing the disease from occurring in an animal that may be predisposed to the disease but does not yet experience or exhibit symptoms of the disease (prophylactic treatment), inhibiting the disease (slowing or arresting its development), providing relief from the symptoms or side-effects of the disease (including palliative treatment), and relieving the disease (causing regression of the disease).

The structure of the polyorthoester useful for the pharmaceutical composition of the present invention, as shown in formula I, formula II, formula III and formula IV, is one of alternating residues of a diketene acetal and a diol, with each adjacent pair of diketene acetal residues being separated by the residue of one polyol, preferably a diol.

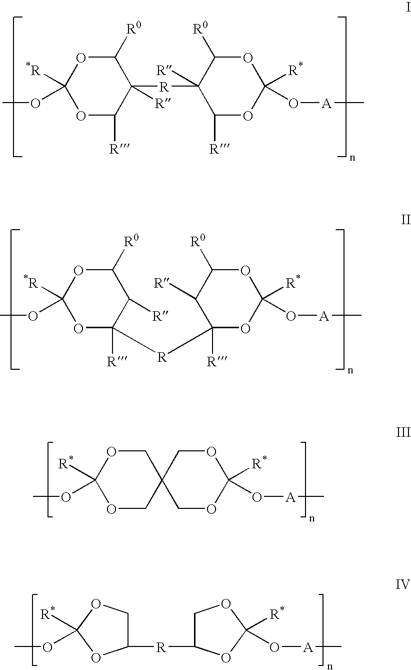

Polyorthoesters having a higher mole percentage of the “o-hydroxy acid containing” units will have a higher rate of bioerodibility. Preferred polyorthoesters are those in which the mole percentage of the “α-hydroxy acid containing” units is at least 0.01 mole percent, in the range of about 0.01 to about 50 mole percent, more preferably from about 0.05 to about 30 mole percent, for example from about 0.1 to about 25 mole percent, especially from about 1 to about 20 mole percent. The mole percentage of the “α-hydroxy acid containing” units appropriate to achieve the desired composition will vary from formulation to formulation.

Preferred polyorthoesters are those where: n is an integer of 5 to 1000; the polyorthoester has a molecular weight of 1000 to 20,000, preferably 1000 to 10,000, more preferably 1000 to 8000; R⁵ is hydrogen or methyl;

R⁶ is: 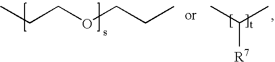
where s is an integer of 0 to 10, especially 1 to 4; t is an integer of 2 to 30, especially 2 to 10; and R⁷ is hydrogen or methyl;

R³ is:

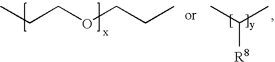

where x is an integer of 0 to 10, especially 1 to 4; y is an integer of 2 to 30, especially 2 to 10; and R⁸ is hydrogen or methyl;

R⁴ is selected from the residue of an aliphatic diol of 2 to 20 carbon atoms, preferably 2 to 10 carbon atoms, interrupted by one or two amide, imide, urea, or urethane groups;

the proportion of units in which A is R¹ is about 0.01-50 mol %, preferably 0.05-30 mol %, more preferably 0.1-25 mol %;

the proportion of units in which A is R² is less than 20%, preferably less than 10%, especially less than 5%, and

the proportion of units in which A is R⁴ is less than 20%, preferably less than 10%, especially less than 5%.

While the presence of any of these preferences results in a polyorthoester that is more preferred than the same polyorthoester in which the preference is not met, the preferences are generally independent, and polyorthoesters in which a greater number of preferences is met will generally result in a polyorthoester that is more preferred than that in which a lesser number of preferences is met.

Preparation of the Polyorthoesters

The Excipients

The excipients suitable for the present invention are pharmaceutically acceptable and polyorthoester-compatible materials. They are liquid at room temperature, and are readily miscible with the polyorthoesters.

Suitable excipients include poly(ethylene glycol) ether derivatives having a molecular weight of between 200 and 4,000, such as poly(ethylene glycol) mono- or di-alkyl ethers, preferably poly(ethylene glycol)monomethyl ether 550 or poly(ethylene glycol)dimethyl ether 250; poly(ethylene glycol)copolymers having a molecular weight of between 400 and 4,000 such as poly(ethylene glycol-co-polypropylene glycol); propylene glycol mono- or di-esters of a C₂₋₁₉ aliphatic carboxylic acid or a mixture of such acids, such as propylene glycol dicaprylate or dicaprate; mono-, di- or tri-glycerides of a C₂₋₁₉ aliphatic carboxylic acid or a mixture of such acids, such as glyceryl caprylate, glyceryl caprate, glyceryl caprylate/caprate, glyceryl caprylate/caprate/laurate, glycofurol and similar ethoxylated tetrahydrofurfuryl alcohols and their C₁₋₄ alkyl ethers and C₂₋₁₉ aliphatic carboxylic acid esters; and biocompatible oils such as sunflower oil, sesame oil and other non- or partially-hydrogenated vegetable oils.

The Delivery Vehicle

The delivery vehicle comprises a polyorthoester and an excipient selected from those described in preceding sections. The concentrations of the polyorthoester and the excipient in the delivery vehicle may vary. For example, the concentration of the excipient in the vehicle may be in the range of 1-99% by weight, preferably 5-80% weight, especially 20-60% by weight of the vehicle.

While the singular form is used to describe the polyorthoester and excipient in this application, it is understood that more than one polyorthoesters and excipients selected from the groups described above may be used in the delivery vehicle. Semi-solid pharmaceutical compositions comprising the active agents and their methods of preparation are disclosed in U.S. Pat. No. 6,861,068 and U.S. patent application Ser. No. 10/953,841, filed Sep. 28, 2004, the disclosures of which are incorporated herein by reference in their entirety. It is also understood that while not required, other pharmaceutically acceptable inert agents such as coloring agents and preservatives may also be incorporated into the composition.

The formulation comprising the semi-solid pharmaceutical composition of the present invention is easily syringable or injectable, meaning that it can readily be dispensed from a conventional tube of the kind well known for topical or ophthalmic formulations, from a needleless syringe, or from a syringe with a 16 gauge or smaller needle (such as 16-25 gauge), and injected subcutaneously, intradermally or intramuscularly. The formulation may be applied using various methods known in the art, including by syringe, injectable or tube dispenser.

After administration by injection, including surface or subcutaneous application, granisetron is released from the composition in a sustained and controlled manner. The rate of release may be regulated or controlled in a variety of ways to accommodate the desired therapeutic effect. The rate may be increased or decreased by altering the mole percentage of the α-hydroxy acid containing units in the polyorthoester, or by selecting a particular excipient, or by altering the amount of the selected excipient, or the combination thereof.

Delivery of Controlled-release Granisetron

The present invention further relates to a method for the treatment or prevention of emesis in a patient which comprises administering a pharmaceutical composition comprising a 5-HT₃ antagonist, wherein the 5-HT₃ antagonist minimize the side effects of nausea and/or emesis associated with other pharmacological agents. In a particularly preferred aspect, the 5-HT₃ antagonist is granisetron.

As used herein, the term “emesis” includes nausea and vomiting. The 5-HT₃ antagonists in the semi-solid injectable form of the present invention are beneficial in the therapy of acute, delayed or anticipatory emesis, including emesis induced by chemotherapy, radiation, toxins, viral or bacterial infections, pregnancy, vestibular disorders (e.g. motion sickness, vertigo, dizziness and Meniere's disease), surgery, migraine, and variations in intracranial pressure. The 5-HT₃ antagonists of use in the invention are of particular benefit in the therapy of emesis induced by radiation and/or by chemotherapy, for example during the treatment of cancer, or radiation sickness; and in the treatment of post-operative nausea and vomiting. The 5-HT₃ antagonists in the semi-solid injectable form of the invention are beneficial in the therapy of emesis induced by antineoplastic (cytotoxic) agents including those routinely used in cancer chemotherapy, and emesis induced by other pharmacological agents, for example, alpha-2 adrenoceptor antagonists, such as yohimbine, MK-912 and MK-467, and type IV cyclic nucleotide phosphodiesterase (PDE4) inhibitors, such as RS14203, CT-2450 and rolipram. In one aspect of each of the above, the 5-HT₃ antagonist is granisetron.

Particular examples of chemotherapeutic agents are described, for example, by D. J. Stewart in Nausea and Vomiting: Recent Research and Clinical Advances, ed. J. Kucharczyk et al., CRC Press Inc., Boca Raton, Fla., USA, 1991, pages 177-203, see page 188. Examples of commonly used chemotherapeutic agents include cisplatin, dacarbazine (DTIC), dactinomycin, mechlorethamine (nitrogen mustard), streptozocin, cyclophosphamide, carmustine (BCNU), lomustine (CCNU), doxorubicin (adriamycin), daunorubicin, procarbazine, mitomycin, cytarabine, etoposide, methotrexate, 5-fluorouracil, vinblastine, vincristine, bleomycin and chlorambucil (see R. J. Gralle et al. in Cancer Treatment Reports, 1984, 68, 163-172).

The semi-solid injectable form of granisetron of the present invention is prepared by incorporating the antiemetic agent into the delivery vehicle in a manner as described above. The concentration of granisetron may vary from about 0.1-80 wt. %, preferably from about 0.2-60 wt. %, more preferably from about 0.5-40 wt. %, most preferably from about 1-5 wt. %, for example, about 2-3 wt. %. The semi-solid composition is then filled into a syringe with a 16-25 gauge needle, and injected into sites that have been determined to be most effective. The semi-solid injectable composition of the present invention can be used for controlled delivery of both slightly soluble and soluble antiemetic agents.

In another aspect of the invention, granisetron may be used in the form of a salt or salts or mixtures of granisetron and the salt of granisetron. Suitable pharmaceutically acceptable salts of granisetron of use in the present invention include acid addition salts which may, for example, be formed by mixing a solution of granisetron with a solution of a pharmaceutically acceptable non-toxic acid such as hydrochloric acid, iodic acid, fumaric acid, maleic acid, succinic acid, acetic acid, citric acid, tartaric acid, carbonic acid, phosphoric acid, sulfuric acid and the like. Salts of the amine group may also comprise the quaternary ammonium salts in which the amino nitrogen atom carries an alkyl, alkenyl, alkynyl or aralkyl group.

The present invention is further directed to a method for ameliorating the symptoms attendant to emesis in a patient comprising administering to the patient 5-HT₃ antagonists. In accordance with the present invention the 5-HT₃ antagonists are administered to a patient in a quantity sufficient to treat or prevent the symptoms and/or underlying etiology associated with emesis in the patient. In one aspect of the above, the 5-HT₃ antagonist is granisetron.

### BRIEF DESCRIPTION OF THE FIGURES

FIG. 1 depicts the incidence of nausea and vomiting following moderately emetogenic chemotherapy follows a well-defined course over time.

FIG. 2 is a schematic of the pharmacokinetics of palonosetron over 168 hours.

FIG. 3 is a schematic of the pharmacokinetics of granisetron following administration of Formulation F over 168 hours.

FIG. 4 depicts a comparison of the pharmacokinetic profile of Formulation F and Aloxi® overlaid against the incidence of vomiting following moderately emetogenic chemotherapy.

### EXPERIMENTAL

Preparation of Formulation:

The various polymers comprising granisetron may be prepared using the procedures as taught herein. In one example, the polymer (Polymer A) comprises 47.4 mole % DETOSU, 42.1 mole % TEG, and 10.5 mole % of the latent acid. The Mw is 5,900-8,100 daltons and the Mn is 2,900-4,000 daltons. In another series of polymer compositions that are prepared, the polymer may comprise of about 40-60 mole % DETOSU, 40-60 mole % TEG, and 5-20 mole % latent acid.

In one example, Formulation F comprises 78.4 weight % Polymer A, 19.6 weight % MPEG 550 (methoxy-polyethylene glycol, Mn 550) and 2% granisetron.

Formulation F comprises of 2% (w/w) granisetron in a proprietary triethylene glycol-poly (ortho ester) (“TEG-POE”) polymer with methoxypolyethylene glycol (“MPEG”) as an excipient to reduce viscosity. The product is supplied as a clear, sterile viscous liquid in pre-filled syringes. Formulation F is manufactured under GMP conditions by dissolving crystalline granisetron in a mixture of MPEG and TEG-POE polymer. Currently the bulk product is sterilized by gamma irradiation and is aseptically filled into syringes.

Formulation F may be administered before a chemotherapy session, preferably about 5 hours before chemotherapy, more preferably about 3 hours before chemotherapy, more preferably about two hours, or about one hour before chemotherapy. Most preferably, Formulation F is administered as a subcutaneous injection, approximately 30 minutes before chemotherapy. Using the method taught herein, a single injection will provide protection against acute and delayed onset of nausea and vomiting.

Pharmacokinetic Study of the Extended Release Formulation:

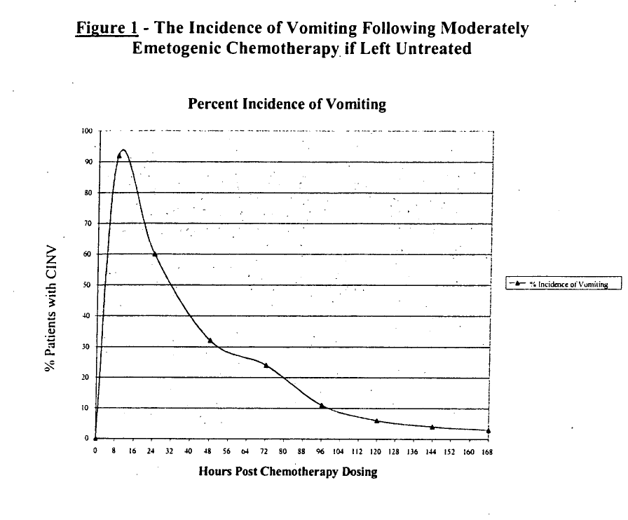

The figures below provide a comparison of the pharmacokinetic profiles of Formulation F and Aloxi® in the context of the timeline of the incidence of CINV. The incidence of nausea and vomiting following moderately emetogenic chemotherapy follows a well-defined course over time (see FIG. 1). As indicated, the incidence of CINV in the acute phase, (less than 24 hours post dosing), is quite marked, with approximately 95% of patients receiving emetogenic chemotherapy suffering an emetic episode in the absence of treatment. All currently approved 5HT₃ receptor antagonists have demonstrated equivalent efficacy in this acute phase of the condition. The delayed phase of CINV, which is not addressed by three of the four products marketed in the U.S., occurs after 24 hours and is largely resolved by 120 hours post dosing (day 5).

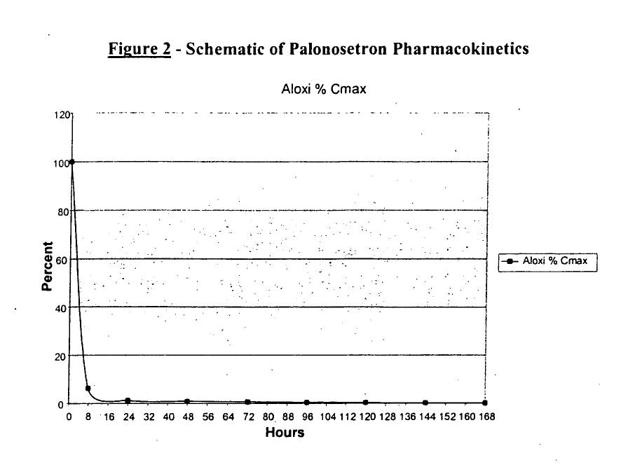

Aloxi® (palonosetron) is the only 5HT₃ antagonist approved for the treatment of acute and delayed CINV. This drug is administered via a slow intravenous infusion. The effect of palonosetron is considered to be due to the very long elimination half life of the drug. Half life values ranging from 43 to 128 hours have been reported. A schematic of palonosetron pharmacokinetics is presented in FIG. 2. The pharmacokinetic profile shows two very distinct phases. The initial distribution phase reduces circulatory levels dramatically and quickly. The secondary elimination phase is the very slow elimination phase reflected in the half life. Aloxi® is still detectable in the systemic circulation at 168 hours or 7 days.

Granisetron has been reported in the literature to be effective for the treatment of delayed onset CINV when administered twice a day. We believe that when this active pharmaceutical ingredient (API) is administered in a specifically designed proprietary formulation having the desired controlled and extended release characteristics, a single dose given on the day of chemotherapy can be expected to provide protection against both acute and delayed onset CINV. Therefore, one goal of the extensive formulation development work that ultimately led to Formulation F and its pharmaceutical formulation was to create a formulation having a pharmacokinetic profile which more closely mirrored the incidence of the CINV. That is, a formulation that provides substantial levels of granisetron over 24 hours and sustained levels over 96 hours, with no drug present beyond the course of condition. In some cases, no drug is present at 144 hours. Such a profile would be potentially more beneficial over the course treatment of the condition. In addition, such a profile would provide optimal therapeutic advantages as the absence of detectable levels of drug when the condition has run its course would reduce the possibility of drug interactions beyond the treatment period, and would minimize the potential side effects that may be caused by the active agent.

Clinical Studies:

We have conducted a multi dose Phase I safety, tolerability and pharmacokinetic study of Formulation F in healthy male volunteers. The primary objective of the Phase I study was to determine the safety and tolerability of ascending single subcutaneous doses of Formulation F in healthy male subjects and to compare the pharmacokinetic profile with the single dose pharmacokinetics of granisetron HCl administered as a subcutaneous injection. The secondary objective was to determine the absolute bioavailability of granisetron following a single dose of subcutaneously administered Formulation F. The trial included four cohorts each of six subjects. Analysis of data from one study of four cohorts of six patients showed that measurable blood levels were achieved over a three to five-day period. A Phase 2 study to evaluate the pharmacokinetics, safety and tolerability of a single dose of Formulation F administered to patients undergoing treatments with moderately emetogenic chemotherapy for cancer has been completed, and the relevant data are provided herein.

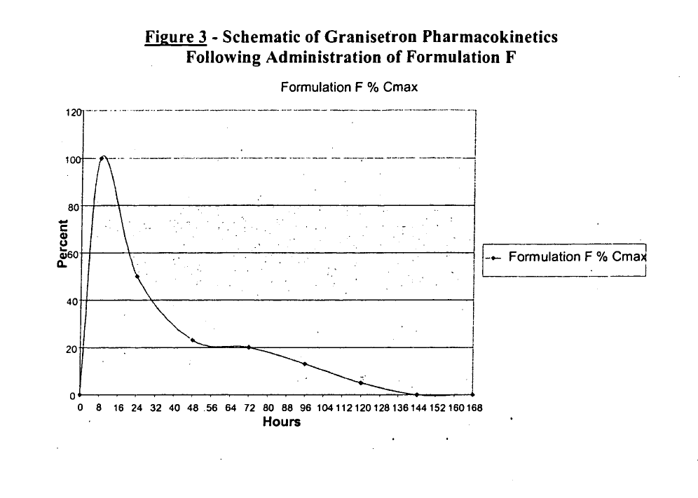

The results of a Phase 1 pharmacokinetic study (see below) in healthy normal volunteers suggest that we achieved the desired plasma profile. A schematic of plasma granisetron concentration following administration of Formulation F using the methods of the present invention is presented in FIG. 3.

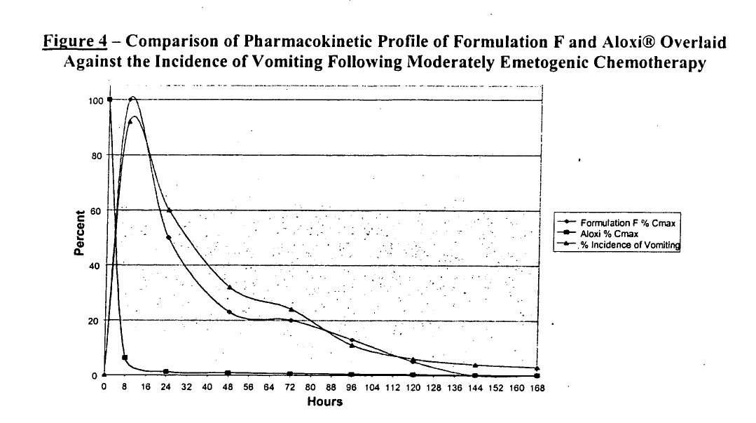

FIG. 4 compares the pharmacokinetic profile of Formulation F (FIG. 3) and Aloxi® (FIG. 2) overlaid against the incidence of CINV (FIG. 1) following the administration of moderately emetogenic chemotherapy. As evident from FIG. 4, we achieved the formulation design goal of matching the pharmacokinetic profile of granisetron in the Formulation F formulation to the incidence of CINV over both the acute and delayed phase.

The release characteristics of granisetron from Formulation F have been evaluated in vitro and the pharmacokinetics (pharmacokinetics) of granisetron in the TEG-POE vehicle have been evaluated in rats and dogs and compared to the pharmacokinetics parameters for granisetron in a saline formulation. The toxicological profile of Formulation F has been evaluated for its local and systemic safety following a single subcutaneous dose in rats and dogs with a follow-up period up to approximately 30 and 15 days, respectively. Repeat dose studies in rat and dog in which Formulation F was administered over 28 days have also been completed.

Data from the in vitro release of granisetron from Formulation F in conjunction with the pharmacokinetics profile of granisetron in rats and dogs when administered in the TEG-POE vehicle indicate that there is sustained release of granisetron. The data, therefore, suggest that the pharmacologic activity of granisetron will be maintained for a longer duration than the duration observed with currently approved commercial formulations of granisetron. The longer duration is expected to provide protection against acute and delayed onset of CINV.

Phase I Study: Following single subcutaneous injections of Formulation F, containing 2.5, 5, 10 and 20 mg of granisetron, respectively, there was a prolonged release of granisetron, with maximum plasma concentrations occurring at a median Tmax of 8 hours. Granisetron could no longer be detected in the plasma of most of the 2.5 and 5 mg subjects by 96 hours; plasma levels were detectable in the 10 mg subjects up to 120 hours and in the 20 mg subjects plasma levels at 140 hours after the injection.

The absolute bioavailability of granisetron following the subcutaneous doses of Formulation F granisetron was approximately 100%. AUC & Cmax values increased in a dose-proportional manner over the 2.5 to 10 mg dose range with an 8-fold increase in dose resulting in an approximate 8-fold and 8-fold increase in AUC & Cmax respectively.

[image]

As has been reported in the literature and in the labeling for Kytril®, the clearance of granisetron in cancer patients is approximately half that of normal volunteers. The labeling for oral Kytril® indicates that when the drug is administered at 1 mg bid over seven days, the peak plasma concentrate was 6.0 ng/ml. This peak level was correlated with a high degree of efficacy: in a study of 233 patients, 81% demonstrated “complete response” over 24 hours (the acute phase). Complete response in this study was defined as no vomiting, no moderate or severe nausea, and no rescue medication.

Based on these data, it can be anticipated that a single dose of Formulation F would deliver therapeutically relevant levels of granisetron into the systemic circulation for a period of 3 to 5 days, which would address both the acute and delayed phase of CINV.

The results of a Phase I study suggested that Formulation F was safe and well tolerated when administered by subcutaneous injection at doses of 125, 250, 500, and 1000 mg. No serious adverse events were reported. The side effects noted were all mild and included erythema, tenderness, and pain at the injection site, but only upon palpation. A small mass was noted at some injection sites and all instances of this were resolved within a few days. All reports of side effects were transient in nature and resolved before the final study visit (14 days following the injection).

A Phase 2 study was conducted to evaluate the pharmacokinetics, safety and tolerability of a single dose of Formulation F administered to patients currently undergoing treatments with moderately emetogenic chemotherapy for cancer is well advanced. This is a multi-center, ascending subcutaneous dose, sequential group study. Thirty evaluable patients were evaluated in three groups, each comprised of 10 patients. Group A received a Formulation F formulation containing 5 mg of granisetron; Group B received a Formulation F formulation containing 10 mg of granisetron and Group C received a granisetron dose that is not to exceed 20 mg of granisetron. The results of the Phase 2 study are provided herein.

Safety and Efficacy of Using TEG-POE Formulations

We have also conducted Phase 2 clinical trials in patients undergoing surgery for inguinal hernia with a TEG-POE formulation of mepivacaine, designated Formulation C. The TEG-POE polymer vehicle for the drug product Formulation C is the same TEG-POE polymer vehicle used in Formulation F. Formulation C was administered directly into the surgical wound prior to suturing. Patients in this study were administered either 5 g or 10 g of TEG-POE polymer formulation. More than 70 patients have been administered 5 g or 10 g Formulation C in Phase I and Phase II studies, and these patients have been monitored for up to 30 days. There have been no reports of serious adverse effects associated with the administration of Formulation C.

Preparation of Pharmaceutical Compositions:

Semi-solid formulation compositions based on Polymer A with 2 weight % granisetron free base and increasing amounts of MPEG 550 were prepared as per the following table:

| | | | MPEG 550 as a | |
|---|---|---|---|---|
| | | | percent of | |
| | | | Polymer A + | Granisetron free |
| | Polymer A | MPEG 550 | MPEG 550 | base |
| Formulation | (weight %) | (weight %) | (weight %) | (weight %) |
| Formulation F1 | 93.1% | 4.9% | 5% | 2.0% |
| Formulation F2 | 88.2% | 9.8% | 10% | 2.0% |
| Formulation F | 78.4% | 19.6% | 20% | 2.0% |
| Formulation F3 | 68.6% | 29.4% | 30% | 2.0% |
| Formulation F4 | 58.8% | 39.2% | 40% | 2.0% |
| Formulation F5 | 49.0% | 49.0% | 50% | 2.0% |
| Formulation F6 | 39.2% | 58.8% | 60% | 2.0% |
| Formulation F7 | 29.4% | 68.6% | 70% | 2.0% |
| Formulation F8 | 19.6% | 78.4% | 80% | 2.0% |
| Formulation F9 | 9.8% | 88.2% | 90% | 2.0% |
| Formulation F10 | 0% | 98.0% | 100% | 2.0% |

In Vitro Release of Active From Semi-solid Pharmaceutical Compositions

In vitro release of granisetron from formulations based on Polymer A and containing increasing amounts of MPEG 550:

The in vitro release of granisetron from Formulations F-10, described in Example above, is shown in the following graphs:

[image]

[image]

The data shows that after the first 24 hours, the rate of release of granisetron is reduced (extended) as the MPEG 550 content (as a percentage of Polymer A and MPEG 550 content) is increased from 5 weight % to 20 weight %. Further increases in the MPEG 550 content in increments of 10 weight % are clearly associated with a corresponding increase in the amount of granisetron released at each time point, i.e. an increase in the rate of release. This continues until 80 weight % MPEG 550, with the two compositions containing 90 weight % and 100 weight % MPEG 550 being essentially indistinguishable from each other all the granisetron is released from these two formulations almost immediately without any apparent control. Thus, the above data shows that for this set of components (Polymer A, MPEG 550, granisetron), some degree of control is achieved in release of the active pharmaceutical ingredient when the formulation contains as little as approximately 20 weight % poly(ortho ester) and 2 weight % of a basic excipient. In this example, this control is gradually increased (with the rate of granisetron release progressively reduced) as the polymer content is increased in the formulations while keeping the basic excipient at 2 weight %.

As the data shows, the composition with 20% MPEG 550, exemplified by Formulation F, has the slowest rate of granisetron release, the composition of which may be particularly useful for clinical trials. In addition, varying degrees of control are achievable as desired by varying the MPEG 550 content in the composition.

Results of Pharmacokinetic Studies:

Protocol Reference (Covance 7436-125 & 126): The blood level data reported for subjects 601, 602, 603, 604, 605, 606, 607, 608, 609, 610, 801, 802, 803, 804, 805, 806 & 807 in study C2005-01 are summarized below. The data for the attached table and charts was obtained from the references noted below.

pK Profile of Chemo Patients Receiving ˜250 mg Formulation Containing ˜5 mg Granisetron (QC'd Plasma Data)
Cohort A

| | Subject | | | | | | | | | | | | | | | |
|---|---|---|---|---|---|---|---|---|---|---|---|---|---|---|---|---|
| TP Hour | 605 | 606 | 607 | 608 | 609 | 610 | 801 | 802 | 803 | 804 | 805 | 806 | 807 | Average | SD | N |
| 0 | 0.300 | 0.300 | 0.300 | 0.300 | 0.300 | 0.300 | 0.300 | 0.300 | 0.300 | 0.300 | 0.300 | 0.300 | 0.300 | 0.300 | 0.000 | 13 |
| 0.5 | 0.728 | 1.81 | 3.16 | 4.42 | 2.72 | 0.922 | 0.922 | 0.976 | 2.23 | 1.54 | 0.320 | 2.07 | 0.300 | 1.701 | 1.210 | 13 |
| 2 | 3.36 | 4.57 | 8.13 | 5.71 | 4.42 | 1.93 | 2.00 | 1.35 | 3.35 | 3.73 | 1.10 | 2.52 | 2.03 | 3.400 | 1.968 | 13 |
| 6 | 6.26 | 14.3 | 10.8 | 5.55 | 7.74 | 4.68 | 5.61 | 2.79 | 4.99 | 8.93 | 4.92 | 6.34 | 16.8 | 7.670 | 4.069 | 13 |
| 24 | 2.54 | 14.1 | 4.86 | 11.4 | 7.78 | 10.8 | 9.23 | 4.13 | 9.41 | 14.2 | 6.29 | 13.8 | 27.9 | 10.495 | 6.486 | 13 |
| 48 | 0.328 | 6.79 | 1.50 | 5.58 | 2.98 | 11.1 | 6.32 | 4.48 | 7.35 | 5.75 | 4.97 | 11.1 | 31.8 | 7.696 | 7.895 | 13 |
| 72 | 0.300 | 1.06 | 0.498 | 2.61 | 0.851 | 9.66 | 2.91 | 2.52 | 2.84 | 2.08 | 1.90 | 4.95 | 23.9 | 4.314 | 6.370 | 13 |
| 96 | 0.300 | 0.593 | 0.300 | 1.89 | 1.08 | 5.66 | 2.10 | 1.09 | 1.77 | 1.24 | 1.09 | 2.53 | 16.2 | 2.757 | 4.270 | 13 |
| 120 | 0.300 | 0.345 | 0.300 | 0.984 | 1.20 | 3.11 | 1.72 | 0.596 | 1.23 | 0.586 | 1.94 | 1.08 | 10.8 | 1.861 | 2.800 | 13 |
| 144 | 0.300 | 0.300 | 0.300 | 0.327 | 0.501 | 1.87 | 1.27 | 0.405 | 0.671 | 0.300 | 1.16 | 0.300 | 8.40 | 1.239 | 2.208 | 13 |
| 168 | 0.300 | 0.300 | 0.300 | 0.300 | 0.300 | 1.80 | 0.944 | 0.300 | 0.300 | 0.300 | 0.587 | 0.300 | 4.19 | 0.786 | 1.111 | 13 |

Reference: Covance pw = pass2word C2005-01 QCd data.XLS

[image]

[image]

[image]

[image]

As evident from the results noted above, the composition comprising granisetron of the present invention provide significant advantages over other antiemetic agents known in the art because the composition provides a long acting formulation that provides an effective treatment for the prevention, reduction or elevation of both acute and delayed emesis using a single dosage.

The foregoing is offered primarily for purposes of illustration. It will be readily apparent to those skilled in the art that the molecular structures, proportions of the various components in the delivery vehicle or pharmaceutical composition, method of manufacture and other parameters of the invention described herein may be further modified or substituted in various ways without departing from the spirit and scope of the invention. For example, effective dosages other than the particular dosages as set forth herein above may be applicable as a consequence of variations in the responsiveness of the mammal being treated for any of the indications with the compounds of the invention indicated above. Likewise, the specific pharmacological responses observed may vary according to and depending upon the particular active compounds selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

What is claimed is:

1. A pharmaceutical composition for the sustained and controlled release of an effective amount of a selective 5-hydroxytryptamine 3 (5-HT₃) receptor antagonist for the prevention, reduction or alleviation of acute and delayed chemotherapy-induced nausea and vomiting (CINV) following a course of emetogenic chemotherapy, wherein:
the composition is administered by subcutaneous injection, the composition comprising a 5-HT₃ receptor antagonist, a semi-solid delivery vehicle and a pharmaceutically acceptable liquid excipient; wherein
the composition, when administered in a single dosage, provides a release profile of the 5-HT₃ receptor antagonist that tracks the profile of an incidence of vomiting; and wherein the composition provides a level of the 5-HT₃ receptor antagonist over 24 hours to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no 5-HT₃ receptor antagonist concentration in plasma at about 144 hours.

2. The pharmaceutical composition of claim 1, wherein the 5-HT₃ receptor antagonist is granisetron.

3. The pharmaceutical composition of claim 1, wherein the effective amount of the 5-HT₃ receptor antagonist is a single dose of about 5 mg to about 10 mg.

4. The pharmaceutical composition of claim 1, wherein the administration by subcutaneous injection is performed at about three hours, two hours, one hour, or 30 minutes before chemotherapy.

5. The pharmaceutical composition of claim 4, wherein the administration by subcutaneous injection is performed at about 30 minutes before chemotherapy.

6. The pharmaceutical composition of claim 1, wherein the composition provides a substantial level of the 5-HT₃ receptor antagonist over 24 hours to provide a % Cₘₐₓ profile that is within 10% of the profile in the incidence of vomiting.

7. The pharmaceutical composition of claim 1, wherein the composition provides sustained levels over 96 hours to provide a % Cₘₐₓ profile that is within 5% of the profile in the incidence of vomiting.

8. The pharmaceutical composition of claim 1, wherein the composition provides a substantial level of the 5-HT₃ receptor antagonist over 24 hours to provide a % Cₘₐₓ profile that is within 10% of the profile in the incidence of vomiting, provides sustained levels over 96 hours to provide a % Cₘₐₓ profile that is within 5% of the profile in the incidence of vomiting, and provides essentially no 5-HT₃ receptor antagonist concentration in plasma at about 144 hours.

9. The pharmaceutical composition of claim 1, wherein the administration of an effective amount of the 5-HT₃ receptor antagonist to a patient results in further reducing the incidence of reported headaches to less than about 40%, 30%, 20% or about 10% in patients receiving chemotherapy.

10. A pharmaceutical composition for the sustained and controlled release of an effective amount of granisetron for the prevention, reduction or alleviation of acute and delayed chemotherapy-induced nausea and vomiting (CINV) following a course of emetogenic chemotherapy, wherein:
the composition is administered by subcutaneous injection, the composition comprising granisetron, a semi-solid delivery vehicle and a pharmaceutically acceptable liquid excipient; wherein:
(A) the semi-solid delivery vehicle, comprises:
(i) a polyorthoester of formula I 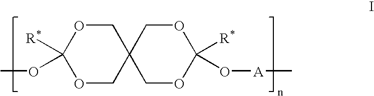
where:
R* is a C₁₋₄ alkyl;
n is an integer of at least 5; and
A is R¹, R², R³, or R⁴, where
R¹ is: 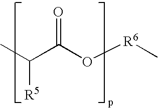
where:
p is an integer of 1 to 20;
R⁵ is hydrogen or C₁₋₄ alkyl, and
R⁶ is: 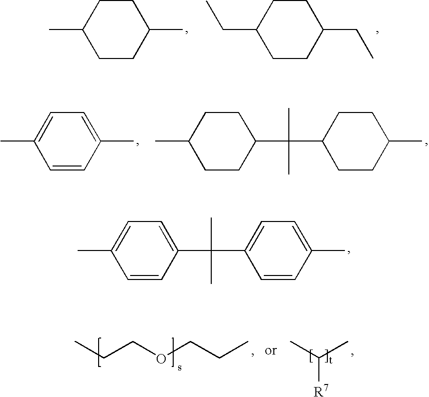
where:
s is an integer of 0 to 30;
t is an integer of 2 to 200; and
R⁷ is hydrogen or C₁₋₄ alkyl;
R² is: 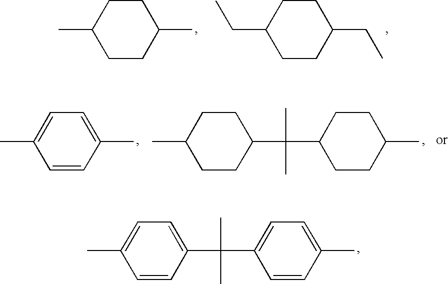
R³ is: 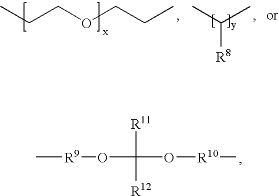
where:
x is an integer of 0 to 30;
y is an integer of 2 to 200;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is C₁₋₆ alkyl; or R¹¹ and R¹² together are C₃₋₁₀ alkylene; and
R⁴ is the residue of a diol containing at least one functional group independently selected from amide, imide, urea, and urethane groups;
in which at least 0.01 mol percent of the A units are of the formula R¹; and
(ii) a pharmaceutically acceptable, polyorthoester-compatible liquid excipient selected from polyethylene glycol ether derivatives having a molecular weight between 200 and 4000, polyethylene glycol copolymers having a molecular weight between 400 and 4000, mono-, di-, or tri-glycerides of a C₂₋₁₉ aliphatic carboxylic acid or a mixture of such acids, alkokylated tetrahydrofurfuryl alcohols and their C₁₋₄ alkyl ethers and C₂₋₁₉ aliphatic carboxylic acid esters, and biocompatible oils; and
(B) wherein the composition, when administered in a single dosage, provides the release profile of granisetron that tracks the profile of the incidence of vomiting, and wherein the composition provides a substantial level of granisetron over 24 hours to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no granisetron concentration in plasma at about 144 hours.

11. The composition of claim 10, wherein:
A is R¹, R³, or R⁴, where
R¹ is: 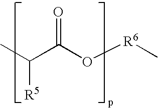
where:
p is an integer of 1 to 20;
R³ and R⁶ are each independently: 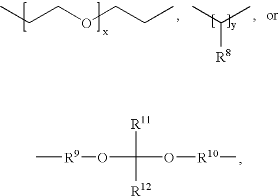
where:
x is an integer of 0 to 30;
y is an integer of 2 to 200;
R⁸ is hydrogen or C₁₋₄ alkyl;
R⁹ and R¹⁰ are independently C₁₋₁₂ alkylene;
R¹¹ is hydrogen or C₁₋₆ alkyl and R¹² is C₁₋₆ alkyl; or R¹¹ and R¹² together are C₃₋₁₀ alkylene;
R⁴ is a diol containing at least one functional group independently selected from amide, imide, urea, and urethane groups; and
R⁵ is hydrogen or C₁₋₄ alkyl; and
in which at least 0.01 mol percent of the A units are of the formula R¹.

12. The composition of claim 10, where the concentration of the polyorthoester ranges from 1% to 99% by weight.

13. The pharmaceutical composition of claim 10, where the polyorthoester is of formula I, where:
none of the units have A equal to R²;
R³ is: 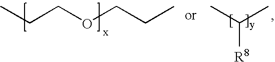
where:
x is an integer of 0 to 10;
y is an integer of 2 to 30; and
R⁶ is: 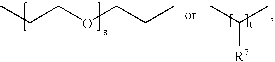
where:
s is an integer of 0 to 10;
t is an integer of 2 to 30; and
R⁵, R⁷, and R⁸ are independently hydrogen or methyl.

14. The pharmaceutical composition of claim 10, where the fraction of granisetron is from 0.1% to 80% by weight of the composition.

15. A method for the prevention, reduction or alleviation of acute and delayed chemotherapy-induced nausea and vomiting (CINV) following a course of emetogenic chemotherapy, the method comprising:
administering a single dosage of a pharmaceutical composition for the sustained and controlled release of an effective amount of a selective 5-hydroxytryptamine 3 (5-HT₃) receptor antagonist by subcutaneous injection; wherein
the composition comprises a 5-HT₃ receptor antagonist, a semi-solid delivery vehicle and a pharmaceutically acceptable liquid excipient, wherein the method provides the release profile of the 5-HT₃ receptor antagonist that tracks the profile of the incidence of vomiting, and wherein
the method provides a substantial level of the 5-HT₃ receptor antagonist over 24 hours to provide a % Cₘₐₓ profile that is within 20% of the profile in the incidence of vomiting, provides a sustained levels over 96 hours to provide a % Cₘₐₓ profile that is within 10% of the profile in the incidence of vomiting, and provides essentially no 5-HT₃ receptor antagonist concentration in plasma at about 144 hours.

16. The method of claim 15, wherein the 5-HT₃ receptor antagonist is granisetron.

17. The method of claim 16, wherein the administration by subcutaneous injection is performed at about three hours, two hours, one hour, or 30 minutes before chemotherapy.

18. The method of claim 16, wherein the composition provides a substantial level of the 5-HT₃ receptor antagonist over 24 hours to provide a % Cₘₐₓ profile that is within 10% of the profile in the incidence of vomiting.

19. The method of claim 16, wherein the composition provides sustained levels over 96 hours to provide a % Cₘₐₓ profile that is within 5% of the profile in the incidence of vomiting.

20. The method of claim 16, wherein the composition provides a substantial level of the 5-HT₃ receptor antagonist over 24 hours to provide a % Cₘₐₓ profile that is within 10% of the profile in the incidence of vomiting, provides a sustained levels over 96 hours to provide a % Cₘₐₓ profile that is within 5% of the profile in the incidence of vomiting, and provides essentially no 5-HT₃ receptor antagonist concentration in plasma at about 144 hours.

21. The method of claim 16, wherein the administration of an effective amount of the 5-HT₃ receptor antagonist to a patient results in further reducing the incidence of reported headaches to less than about 40%, 30%, 20% or about 10% in patients receiving chemotherapy.

22. The method of claim 16, wherein the administration of an effective amount of the 5-HT₃ receptor antagonist to a patient results in a statistically significant reduction in the incidence of vomiting following emetogenic chemotherapy than that of the administration of palonosetron by iv infusion.
